(19) United States
(12) Patent Application Publication
(10) Pub. No.: US 2023/0340605 A1
(43) Pub. Date: Oct. 26, 2023

(54) BIOMARKERS PREDICTING CLINICAL RESPONSE OF A VEGF-A INHIBITORY DRUG IN CANCER PATIENTS, METHOD FOR THEIR SELECTION AND USE

(71) Applicants: Oslo Universitetssykehus HF, Oslo (NO); Universitet i Oslo, Oslo (NO); University of Texas, Austin, TX (US)

(72) Inventors: Gunhild M. Mælandsmo, Oslo (NO); Mads Haugland Haugen, Oslo (NO); Olav Engebråten, Oslo (NO); Ole Christian Lingjærde, Oslo (NO); Gordon Mills, Portland, OR (US)

(21) Appl. No.: 17/796,392

(22) PCT Filed: Jan. 28, 2021
(86) PCT No.: PCT/EP2021/052016

§ 371 Date: Jul. 29, 2022

Related U.S. Application Data
(60) Provisional application No. 62/969,770, filed on Feb. 4, 2020.

(51) Int. Cl. C07K 16/22; A61P 35/00; C12Q 1/6886; G01N 33/574

(52) U.S. Cl. CPC C12Q 1/6886; A61P 35/00; C07K 16/22; G01N 33/57415; A61K 2039/505; C07K 2317/24; C12Q 2600/106; C12Q 2600/158

(57) ABSTRACT

The present disclosure relates to biomarkers for predicting clinical response of a VEGF-A (vascular endothelial growth factor A) inhibitory drug in cancer therapy. In particular, the disclosure provides a multi-gene expression signature score named ViRP (VEGF inhibitory Response Predictor) able to predict response to a VEGF-A inhibitory drug in a patient being diagnosed with a solid cancerous tumor.

### FIELD OF THE INVENTION

The present disclosure relates to biomarkers for predicting clinical response of a VEGF-A (vascular endothelial growth factor A) inhibitory drug in cancer therapy.

### BACKGROUND

Treatment of solid tumors and prevention of metastasis through anti-angiogenic treatment has been explored for several decades (Folkman J, Anti-angiogenesis: new concept for therapy of solid tumors. Annals of Surgery, 1972, vol.175, page. 409-416; Kerbel, RS, Tumor Angiogenesis, New England Journal of Medicine, 2008, vol. 358, page 2039-2049).

Vascular Endothelial Growth Factor A (VEGF-A) is an important signaling molecule involved in angiogenesis. The importance of this pathway in cancer cell growth and metastasis has led to the development of angiogenic inhibitory agents for use in cancer therapy. An example of an anti-angiogenic agent is the monoclonal anti-VEGF-A antibody bevacizumab. Bevacizumab is sold under the tradenames Avastin (Roche), Mvasi (Amgen) or Zirabev (Pfizer). Bevacizumab blocks the binding of the circulating VEGF-A to its receptors.

Many patients diagnosed with breast cancer, colorectal-, lung-, ovarian- or kidney cancer benefit from anti-VEGF-A therapies such as treatment with bevacizumab (Roviello G, Bachelot T, Hudis CA, et al: The role of bevacizumab in solid tumours: A literature based meta-analysis of randomised trials. European Journal of Cancer, 2017, vol. 75,p.245-258).

However, different studies have shown that not all patients benefit from treatment with a VEGF-A-inhibitory drug such as bevacizumab. For example, bevacizumab treatment of metastatic breast cancer patients (Miller K, Wang M, Gralow J, et al: Paclitaxel plus Bevacizumab versus Paclitaxel Alone for Metastatic Breast Cancer. New England Journal of Medicine 2007, vol.357, page 2666-2676), metastatic colorectal cancer patients (A. Passardi, et al., Effectiveness of bevacizumab added to standard chemotherapy in metastatic colorectal cancer: final results for first-line treatment from the ITACa randomized clinical trial Annals of Oncology, Volume 26, Issue 6, June 2015, Pages 1201-1207), or in women with newly diagnosed ovarian, fallopian tube, or primary peritoneal carcinoma (Tewari KS et al. Final Overall Survival of a Randomized Trial of Bevacizumab for Primary Treatment of Ovarian Cancer. J Clin Oncol. 2019 Sep 10;37(26):2317-2328). Thus, the clinical use of bevacizumab in breast cancer for example is limited.

Despite this, usefulness in many individual patients is observed, and several studies point to the urgent need for novel biomarkers to select patient sub-populations in which adequate clinical benefit can be achieved, see for example Maru D, Venook AP, Ellis LM: Predictive Biomarkers for Bevacizumab: Are We There Yet? Clinical Cancer Research, 2013, vol.19, p.2824-2827 or Gampenrieder SP, Westphal T, Greil R: Antiangiogenic therapy in breast cancer. Magazine of European Medical Oncology, 2017, vol.10, p.194-201.

One of the most evident and biological reasonable biomarkers is the plasma VEGF-A, and though promising in some studies, evidence from the latter MERiDiAN trial did not support its use for identifying patients with benefit from added bevacizumab (Miles D, Cameron D, Bondarenko I, et al: Bevacizumab plus paclitaxel versus placebo plus paclitaxel as first-line therapy for HER2-negative metastatic breast cancer (MERiDiAN): A double-blind placebo-controlled randomised phase III trial with prospective biomarker evaluation. European Journal of Cancer, 2017, vol.70, page146-155).

In the more recent past other elaborate biomarkers at various molecular levels including SNPs in VEGF (Schneider BP, Wang M, Radovich M, et al: Association of Vascular Endothelial Growth Factor and Vascular Endothelial Growth Factor Receptor-2 Genetic Polymorphisms With Outcome in a Trial of Paclitaxel Compared With Paclitaxel Plus Bevacizumab in Advanced Breast Cancer: ECOG 2100. Journal of Clinical Oncology 26:4672-4678, 2008), soluble carbonic anhydrase IX (Janning M, Müller V, Vettorazzi E, et al: Evaluation of soluble carbonic anhydrase IX as predictive marker for efficacy of bevacizumab: a biomarker analysis from the GeparQuinto phase III neoadjuvant breast cancer trial. International Journal of Cancer), BRCA½ mutations (Fasching PA, Loibl S, Hu C, et al: BRCA½ Mutations and Bevacizumab in the Neoadjuvant Treatment of Breast Cancer: Response and Prognosis Results in Patients With Triple-Negative Breast Cancer From the GeparQuinto Study. Journal of Clinical Oncology, 2018, vol. 36, page 2281-2287), Endothelin-1 genetic polymorphism (Gampenrieder SP, Hufnagl C, Brechelmacher S, et al: Endothelin-1 genetic polymorphism as predictive marker for bevacizumab in metastatic breast cancer. The Pharmacogenomics Journal, 2016, vol. 17, page 344) and DNA methylation signatures (Gampenrieder SP, Rinnerthaler G, Hackl H, et al: DNA Methylation Signatures Predicting Bevacizumab Efficacy in Metastatic Breast Cancer. Theranostics, 2018, vol.8, page 2278-2288) have been investigated.

WO 2014/087294 A2 discloses biomarker information based on expression levels of the biomarkers from a baseline sample of a breast tumor of a patient acquired before initiating a breast cancer therapy regimen with bevacizumab compared to response gene expression levels information from a response sample of the breast tumor acquired after initiating the breast cancer therapy regimen by administering a first dose of bevacizumab to the patient. A method to predict outcome of a treatment where all patients must undergo treatment with an initial dose of the antibody in order to determine if the cancer patient will gain a beneficial respond to the treatment or not is expensive and less efficient from a clinical point of view. In addition, treatment with bevacizumab alone and in combination with chemotherapy has been reported to give adverse side effects for some patient (Ranpura, P et al., Treatment-related mortality with bevacizumab in cancer patients - A meta-analysis, JAMA, 2011, Vol.350, No.5, page 487-494), thus making this method less favorable.

It is therefore an urgent need for improved methods for selecting biomarkers and biomarkers that can be used in a routine laboratory setting predicting clinical effect of a VEGF-A inhibitory drug in cancer patients.

### SUMMARY

The present invention is defined by the patent claims. Further optional features of the present invention and definition of terms are disclosed in the detailed description below.

The present disclosure provides in a first aspect an in vitro method for predicting whether a subject diagnosed with a solid malignant tumor is responsive to a VEGF-A (vascular endothelial growth factor A) inhibitory drug, wherein the method comprises the steps:
a. providing a sample comprising cancer cells obtained from the subject;
b. analyzing the sample from step a. by measuring an expression level of at least two genes selected from SYK, NOTCH1, ACACA/ACACB, TP53BP1, CDKN1A, CHEK1, BCL2, MYH9, FN1 and NDRG1;
c. calculating a ViRP (VEGF inhibition Response Predictor) score based on the expression level of the selected genes in step b.; and
d. comparing said ViRP-score calculated in step c. to a selected cutoff ViRP-value,
wherein a ViRP-score being lower than the selected cutoff ViRP-value is predicting that the subject is responsive to a VEGF-A inhibitor drug.

In one embodiment of the first aspect, said expression level is measured for at least three, four, five, six, seven or eight genes selected from the genes in step b.

In one embodiment of the first aspect, said expression level is measured for nine genes selected from the genes in step b.

In one embodiment of the first aspect, said expression level is measured for all ten genes selected from the genes in step b.

In one embodiment of the first aspect said expression level is measured for at least the two genes SYK and MYH9.

In one embodiment of the first aspect said expression level is measured for at least two of the genes selected from the group consisting of SYK, MYH9, NDRG1, CDKN1A and TP53BP1.

In one embodiment of the first aspect said expression level is measured for at least SYK and MYH9 and one or more of the genes selected from NDRG1, CDKN1A and TP53BP1.

In one embodiment of the first aspect said expression level is measured for the five genes SYK, MYH9, NDRG1, CDKN1A and TP53BP1.

In one embodiment of the first aspect, said expression levels are measured by mRNA or protein.

In one embodiment of the first aspect, said expression levels of said genes are normalized.

In one embodiment of the first aspect, said selected normalized cutoff value is from about 40 to about 50.

In one embodiment of the first aspect, said ViRP-score is calculated as the weighted sum of the expression levels adjusted with the respective coefficients in Table 1.

In one embodiment of the first aspect, said ViRP-score is calculated as the weighted sum of the normalized gene expression levels and adding a constant to generate a ViRP-score, wherein

$$V i R P = {A}_{0} + {\sum }_{i = 1}^{n} {A}_{i} ∗ M o l e c u l {e}_{i} ,$$

where n= is the number of genes, A₀ = 0.32, Moleculeᵢ is the normalized expression level of each gene with corresponding Aᵢ coefficients as found in Table1.

In one embodiment of the first aspect, said the VEGF-A inhibitory drug is an anti-VEGF-A antibody.

In one embodiment of the first aspect, said anti-VEGF-A antibody is bevacizumab. In one embodiment of the first aspect, said malignant tumor is a primary malignant tumor or a metastatic malignant tumor selected from the group consisting of breast cancer, colorectal cancer, lung cancer, ovarian cancer, glioblastoma or kidney cancer.

In one embodiment of the first aspect, said malignant tumor is a primary breast cancer or a metastatic breast cancer.

In one embodiment of the first aspect, said sample is a lysate of blood cells comprising cancer cells or a lysate of cells obtained from a malignant tumor biopsy.

In one embodiment of the first aspect, said subject has been diagnosed with breast cancer and is eligible for treatment with neoadjuvant chemotherapy.

The present disclosure provides in a second aspect a method for treatment of a solid malignant tumor in a subject comprising the steps:
a. providing a sample comprising cancer cells obtained from the subject;
b. analyzing the sample from step a. by measuring an expression level of at least two genes selected from SYK, NOTCH1, ACACA/ACACB, TP53BP1, CDKN1A, CHEK1, BCL2, MYH9, FN1 and NDRG1;
c. calculating a ViRP (VEGF inhibition Response Predictor) score based on the expression level of the selected genes in step b.; and
d. administering a therapeutically efficient amount of a VEGF-A inhibitory drug to said subject if the calculated ViRP-score is lower than a selected cutoff ViRP-value.

In one embodiment of the second aspect, said expression level is measured for at least three, four, five, six, seven or eight genes selected from the genes in step b.

In one embodiment of the second aspect, said expression level is measured for nine genes selected from the genes in step b.

In one embodiment of the second aspect, said expression level is measured for all ten genes selected from the genes in step b.

In one embodiment of the second aspect, said expression levels are measured by mRNA or protein.

In one embodiment of the second aspect, said expression levels of said genes are normalized.

In one embodiment of the second aspect, said selected normalized cutoff value is from about 40 to about 50.

In one embodiment, said ViRP-score is calculated as the weighted sum of the expression levels adjusted with the respective coefficients in Table 1.

In one embodiment of the second aspect, said ViRP-score is calculated as the weighted sum of the normalized gene expression levels and adding a constant to generate a ViRP-score, wherein

$$V i R P = {A}_{0} + {\sum }_{i = 1}^{n} {A}_{i} ∗ M o l e c u l {e}_{i} ,$$

where n= is the number of genes, A₀ = 0.32, Moleculeᵢ is the normalized expression level of each gene with corresponding Aᵢ coefficients as found in Table1.

In one embodiment of the second aspect, said VEGF-A inhibitory drug is an anti-VEGF-A antibody.

In one embodiment of the second aspect, said anti-VEGF-A antibody is bevacizumab.

In one embodiment of the second aspect, said malignant tumor is a primary malignant tumor or a metastatic malignant tumor selected from the group consisting of breast cancer, colorectal cancer, lung cancer, ovarian cancer, glioblastoma or kidney cancer.

In one embodiment of the second aspect, said malignant tumor is a primary breast cancer or a metastatic breast cancer.

In one embodiment of the second aspect, said sample is a lysate of blood cells comprising cancer cells or a lysate of cells obtained from a malignant tumor biopsy.

In one embodiment of the second aspect, said subject has been diagnosed with breast cancer and is eligible for treatment with neoadjuvant chemotherapy.

The present disclosure provides in a third aspect a kit for use in the method of the first aspect, wherein the kit comprises reagents for the measuring of the protein expression levels and/or mRNA expression levels of at least two, three, four, five, six, seven, eight, nine or ten genes from the set of genes comprising SYK, NOTCH1, ACACA/ACACB, TP53BP1, CDKN1A, CHEK1, BCL2, MYH9, FN1 and NDRG1.

The present disclosure provides in a fourth aspect a novel method for identifying other predictive signatures, see FIG. 11.

The present model has been trained using a continuous response evaluation of the treatment in contrast to the dichotomous pathological complete response (pCR) vs. non-complete response. This, in combination with the use of a panel of pre-selected proteins of relevance to breast cancer, represents a novel approach. In particular, the closer proximity of proteins to tumor phenotype represents a superior prospect for developing models of biological and clinical relevance than the more widely used mRNA expression. Accordingly, there is also provided a method for identifying other predictive signatures comprising the steps:
a. obtain samples comprising cancer cells
b. screen for aberrant expression on protein level of cancer relevant proteins in the samples
c. select a subset of proteins with a detectable dynamic expression and aberrant expression from step b.
d. train the LASSO regression model by a continuous variable.

The present invention provides in a fifth aspect a composition comprising a VEGF inhibitor drug for use in treatment of a subject diagnosed with a solid malignant tumor and having a ViRP-score that is lower than a selected cutoff ViRP-value.

In one embodiment of the fifth aspect the ViRP-score and the ViRP-value is calculated according to the method in the first aspect.

### FIGURES

FIG. 1. CONSORT diagram

FIG. 2. Mixed distribution of protein variance.

Mixed distribution of protein expression variances, only proteins with variance above intersection line were selected for input in Lasso regression to determine the ViRP signature.

FIG. 3. ViRP-score from protein expression in relation to relative tumor size, pCR and RCB in patients treated with Bev+CTx.
(A) ViRP-score of each patient with corresponding relative tumor size after end of treatment.
(B) ViRP-scores in pCR and non-pCR patients.
(C) ViRP-scores in RCB low (0 and I) and high (II and III) patients.

FIG. 4. Validation with mRNA ViRP-score as proxy for the protein ViRP-score.
(A) Correlation between protein and mRNA VIRP-scores.
(B) mRNA ViRP-scores in pCR and non-pCR patients in the NeoAva cohort.
(C) mRNA ViRP-scores in pCR and non-pCR patients in the PROMIX cohort.

FIG. 5. Predictive performance of the ViRP-score.
(A) ROC curve for ViRP-score prediction of pCR.
(B) ViRP-score prediction of probability for pCR (solid line) with 95% CI (contour). True response marked with on bottom (pCR) and on top (non-pCR).
(C) ROC curve for ViRP-score prediction of low RCB
(D) ViRP-score prediction of probability for low RCB (solid line) with 95% CI (contour). True response marked with on bottom (low RCB) and on top (high RCB).

FIG. 6. ROC curves for prediction of pCR with mRNA ViRP-scores in the NeoAva and PROMIX trials
(A) ROC curve for mRNA VIRP-score prediction of pCR in NeoAva.
(B) ROC curve mRNA VIRP-score prediction of pCR in PROMIX.

FIG. 7. Increase in response rate by selecting patients eligible for Bev+CTx treatment using the ViRP-score.
(A) Fraction of patients with pCR in an unselected population versus selection based on ViRP-score (Fisher’s exact test).
(B) Fraction of patients with low RCB in an unselected population versus selection based on ViRP-score (Fisher’s exact test).

FIG. 8. Example of a minimal signature based on two genes selected from the original ten genes in ViRP. Two-gene based ViRP score from protein expression in relation to relative tumor size, pCR and RCB in patients treated with Bev+CTx.
(A) two-gene based ViRP score of each patient with corresponding relative tumor size after end of treatment.
(B) two-gene based ViRP-scores in pCR and non-pCR patients.
(C) two-gene based ViRP-scores in RCB low (0 and I) and high (II and III) patients.

FIG. 9. Validation with mRNA as proxy for the two-gene based ViRP score.
(A) Correlation between protein and mRNA two-gene based VIRP-scores.
(B) two-gene based mRNA ViRP-scores in pCR and non-pCR patients in the NeoAva cohort.
(C) two-gene based mRNA ViRP-scores in pCR and non-pCR patients in the PROMIX cohort.

FIG. 10. Predictive performance of the two-gene based ViRP-score.
(A) ROC curve for two-gene based ViRP-score prediction of pCR.
(B) two-gene based ViRP-score prediction of probability for pCR (solid line) with 95% CI (contour). True response marked with on bottom (pCR) and on top (non-pCR).
(C) ROC curve for two-gene based ViRP-score prediction of low RCB
(D) two-gene based ViRP-score prediction of probability for low RCB (solid line) with 95% CI (contour). True response marked with on bottom (low RCB) and on top (high RCB).

FIG. 11. Flow chart for method to identify predictive signatures.

FIG. 12. Ranking the relative importance of the ten original genes using the R-package “Relaimpo” demonstrate that some genes may contribute more to the predictive ability to significantly separate the pCR from non-pCR responding patients

### DETAILED DESCRIPTION

The present disclosure provides an in vitro method for predicting whether a subject diagnosed with a solid malignant tumor is responsive to a VEGF-A (vascular endothelial growth factor A) inhibitory drug.

The term “method for predicting” refers to a method that allows determining with a high level of probability (statistically significant), prior to treatment, whether a patient will respond to said treatment.

As used herein, a “responsive to VEGF-A inhibitory drug” or equally a “clinical response to VEGF-A inhibitory drug” is observed when at least one of the symptoms of the cancer to be treated by said VEGF-A inhibitory drug is decreased in a patient after treatment as compared to prior to the treatment. A skilled person is able to determine the response criteria according to the response evaluation criteria for solid tumors that has been defined in a set of rules: Response evaluation criteria in solid tumors (RECIST) is a set of published rules that define when tumors in cancer patients improve (“respond”), stay the same (“stabilize”), or worsen (“progress”) during treatment. The criteria were published in February 2000 by an international collaboration including the European Organization for Research and Treatment of Cancer (EORTC), National Cancer Institute of the United States, and the National Cancer Institute of Canada Clinical Trials Group. Today, the majority of clinical trials evaluating cancer treatments for objective response in solid tumors use RECIST. These criteria were developed and published in February 2000, and subsequently updated in 2009.

Samples comprising cancer cells can be obtained from the subject, i.e. a patient diagnosed with a solid malignant tumor. The solid malignant tumor may be a primary malignant tumor, or a metastatic malignant tumor selected from the group consisting breast cancer, colorectal cancer, lung cancer, ovarian cancer, glioblastoma or kidney cancer. In some embodiments, the solid malignant tumor is primary breast cancer, locally advanced breast cancer or a metastatic breast cancer. In some embodiments, the solid malignant tumor may be a HER2 positive malignant tumor. In some embodiments of the invention the solid malignant tumor may be a HER2 negative malignant tumor. In some embodiments, the solid malignant tumor is primary breast cancer, or a metastatic breast cancer furthered characterized as being HER2 negative.

The sample may be collected in any clinically acceptable manner, which will ensure that gene-specific polynucleotides (i.e. transcript RNA or mRNA) or proteins are preserved. The sample may comprise any clinically relevant tissue such as a tumor biopsy in form of a tumor needle biopsy, formalin-fixed paraffin-embedded tumor section or a frozen tumor tissue. The sample may contain a mixture of tumor and stromal cells. The sample can be a body fluid comprising tumor cells, for example be blood, plasma, serum, ascitic or cystic fluid, urine or nipple exudate.

The samples can be analyzed by measuring an expression level of at least two genes selected from SYK, NOTCH1, ACACA/ACACB, TP53BP1, CDKN1A, CHEK1, BCL2, MYH9, FN1 and NDRG1.

Measuring the expression levels of said genes may be done by well-known mRNA quantification methods or protein quantification methods. In the present disclosure, the expression level may be quantified by measuring mRNA or protein levels directly or indirectly from cell lysates made from the above-mentioned samples.

Examples of suitable mRNA quantification methods include, without limitation, hybridization-based assays, such as microarray analysis and similar formats (e.g., Whole Genome DASL Assay, Illumina, Inc., San Diego, CA), polymerase-based assays, such as RT-PCR (e.g.,TAQMAN®), or real time quantitative reverse transcription PCR (real time qRT-PCR), (e.g., as commercialized by Invitrogen; or Life Technologies), flap-endonuclease-based assays (e.g., INVADER® assay), as well as multiplex assays involving direct RNA (mRNA) capture with branched DNA (QUANTIGENE® ViewRNA, Affymetrix, Santa Clara, CA), HYBRID CAPTURE® (Digene, Gaithersburg, MD), or NCOUNTER® Analysis System (NanoString) as described further herein.

Examples of suitable protein quantification methods includes standard immunoassays, e.g., ELISA, Western blot, or RIA assay. Antibodies specific for the proteins encoded by the genes in the gene signatures described herein, Table 3, can be used for detection and quantification of proteins by one of a number of suitable immunoassay methods that are well known in the art, such as, for example, those presented in Harlow and Lane (Antibodies: A Laboratory Manual, Cold Spring Harbor Laboratory, New York, 1988, and later editions thereof). Specific antibodies directed to the proteins encoded by the genes of the disclosed gene signatures can also be generated using standard methods known to the skilled in the art.

Quantitative mass spectroscopic (MS) methods, such as Tandem Mass tag (TMT)-MS (Zhang L and Elias JE, Relative Protein Quantification Using Tandem Mass Tag Mass Spectrometry. Methods Mol Biol. 2017;1550:185-198), selected reaction monitoring (SRM) (Wang Q, Chaerkady R, Wu J, et al Mutant proteins as cancer-specific biomarkers Proc Natl Acad Sci U S A. 2011 Feb 8; 108(6): 2444-2449) or surface-enhanced laser desorption-ionization (SELDI), can be used to analyze protein expression in a cancer or tumor tissue or cell sample, as well as non-cancerous cells or tissue, and cells or tissue from a cancer-free subject. SELDI is a solid phase method for desorption in which the analyte is presented to the energy stream on a surface that enhances analyte capture or desorption In one example, SELDI time-of-flight (SELDI-TOF) mass spectrometry is used to detect protein expression, for example, by using the ProteinChip™ (Ciphergen Biosystems, Palo Alto, CA). Alternatively, antibodies are immobilized onto the chromatographic surface using an Fc binding support, or bacterial Fc binding support. Thereafter, the surface is incubated with a sample, such as a cancer sample, and the antibodies on the surface can recognize and bind the antigens present in the sample. Unbound proteins and mass spectrometric interfering compounds are washed away, and the proteins that are bound by antibody and retained on the chromatographic surface are analyzed and detected, such as by SELDI-TOF. The Mass Spectrometry profile from the sample can be compared using differential protein expression mapping, wherein relative expression levels of proteins at specific molecular weights are compared by a variety of statistical techniques and bioinformatic software systems.

Protein levels or phosphorylated protein levels may alternatively be measured by using Reverse Phase Protein Arrays (RPPA) (Lu Y, Ling S, Hegde AM, et al: Using reverse-phase protein arrays as pharmacodynamic assays for functional proteomics, biomarker discovery, and drug development in cancer. Seminars in Oncology 43:476-483, 2016) or the NanoString technology (Lee J, Geiss GK, Demirkan G et al. Implementation of a Multiplex and Quantitative Proteomics Platform for Assessing Protein Lysates Using DNA-Barcoded Antibodies. Molecular & Cellular Proteomics 2018 17 (6) 1245-1258).

The expression levels may be absolute or relative. However, methods for normalizing measured values are well known for skilled persons. Typically, expression levels are normalized with respect to an “endogenous control”. An “endogenous control” as used herein relates to a gene expression product whose expression levels do not change or change only in limited amounts in tumor cells with respect to non-tumorigenic cells. “Endogenous control” is usually the expression product from a housekeeping gene and which codes for a protein which is constitutively expressed and carries out essential cellular functions.

Alternatively, global normalization or normalization against a geometric mean of the expression level of all genes analyzed may be used, in which expression of each gene in the gene signature is normalized against the geometric mean of a larger population or number of assayed genes. As is appreciated by the skilled person in the art, normalization, particularly for microarray assay platforms, is conventionally performed to adjust for effects arising from variation in the microarray technology, rather than from biological differences between the samples, such as RNA samples, or between the addressable probes. In general, global normalization in microarray provides a solution for adjusting for errors that effect entire arrays by scaling the data so that the average measurement is the same for each array (and each color). Scaling is typically accomplished by computing the average expression level for each array, calculating a scale factor equal to the desired average, divided by the actual average, and multiplying every measurement from the array by that scale factor. The desired average can be arbitrary, or it may be calculated from the average of a group of arrays.

Normalizing the expression level of the genes against fragments per kilobase of transcript per million reads (FPKM) is an alternative normalization method primarily used in RNAseq known to a person skilled in the art see for example Ali Mortazavi, Brian A Williams, Kenneth McCue et al. Mapping and quantifying mammalian transcriptomes by RNA-Seq, Nature Methods volume 5, pages 621-628 (2008).

The present disclosure provides a multi-gene expression signature, named ViRP (VEGF inhibitory Response Predictor) which is based on the measured protein or mRNA expression levels of selected genes from Table 1. Notably, three of the proteins in Table 1 are measured in a phosphorylated state: ACC pS79; Myosin IIa pS1943; NDRG1_pT346.

A ViRP-score may be calculated as the weighted sum of the normalized expression level calculated from log2 of the relative expression of each Moleculeᵢ divided by a factor SD defined in Table 2 by the following equation:

$$V i R P = {A}_{0} + {\sum }_{i = 1}^{n} {A}_{i} ∗ M o l e c u l {e}_{i}$$

Where A₀ = 0.32

Moleculeᵢ is the normalized expression level of each protein, phosphorylated protein or mRNA with corresponding Aᵢ coefficients found in Table 1.

TABLE 1

List of ViRP signature proteins and genes encoding said proteins with weights

| | Protein | Gene | Aᵢ coefficient |
|---|---|---|---|
| 1 | ACC_pS79 | ACACA/ACACB | 0.12 |
| 2 | Bc12 | BCL2 | 0.03 |
| 3 | Chk1 | CHEK1 | -0.19 |
| 4 | Fibronectin | FN1 | 0.15 |
| 5 | Myosin_IIa_pS1943 | MYH9 | 0.11 |
| 6 | NDRG1_pT346 | NDRG1 | -0.07 |
| 7 | Notch1 | NOTCH1 | -0.03 |
| 8 | p21 | CDKN1A | 0.00 |
| 9 | Syk | SYK | -0.12 |
| 10 | TP53BP1 | TP53BP1 | 0.08 |

The ViRP-score can thus be used to predict response to a VEGF-A inhibitory drug in a patient being diagnosed with a solid cancerous tumor. A ViRP-score obtained from a sample from a cancer patient, lower than a selected cutoff ViRP-value is indicating that the patient will have a beneficial clinical response to a VEGF-A inhibitory drug.

The genes ACACA and ACACB both encode an acetyl-CoA carboxylase (ACC) and when measuring mRNA levels, this could either be done by measuring the expression from ACACA or ACACB. ACACA/ACACB thus defined in this application as either measuring the expression from ACACA or ACACB.

The measured level of either protein, phosphorylated protein or mRNA levels may be normalized before they can be put into the ViRP-scoring equation.

Moleculeᵢ may be determined by dividing the measured level by the Geometric Mean of protein expression levels of at least two of all ten proteins. Moleculeᵢ is then log2 transformed and divided by the SD factor defined in Table 2, and the ViRP-score calculated using the formula with Aᵢ coefficients (i.e. beta-coefficients) as listed in Table 1.

Moleculeᵢ can for example be determined by dividing the counts by the expression level of a single housekeeping protein selected from a list comprising H3F3A, H3F3B, GAPDH, TUBA1A, TUBA1B, TUBA1C, TUBA3C, TUBA3D, TUBA3D, HNRNPL, PCBP1, RER1, ACTG1, RPS23, ACTB, RPS13, TPT1, ATP5B, CFL1, RPL13A, UBC or UBB. Moleculeᵢ is then log2 transformed and divided by the SD factor defined in Table 2, and the ViRP-score calculated using the formula with Aᵢ weights as listed in Table 1.

Moleculeᵢ can be determined by dividing the counts by the expression of an average of two or more housekeeping genes selected from a list comprising H3F3A, H3F3B, GAPDH, TUBA1A, TUBA1B, TUBA1C, TUBA3C, TUBA3D, TUBA3D, HNRNPL, PCBP1, RER1, ACTG1, RPS23, ACTB, RPS13, TPT1, ATP5B, CFL1, RPL13A, UBC or UBB. Moleculeᵢ is then log2 transformed and divided by the SD factor defined in table 2, and the ViRP-score calculated using the formula with Aᵢ weights as listed in Table 1.

In another embodiment of the method, mRNA expression level corresponding to at least two of the ten genes (Table 1) can be used as a proxy for Moleculeᵢ. Moleculeᵢ can be determined by first correcting the background using negative probes included, then normalize using positive probes included. Moleculeᵢ is then log2 transformed and divided by the SD factor defined in Table 2, and the ViRP-score calculated using the formula with Aᵢ weights as listed in Table 1.

In another embodiment of the method mRNA expression level of at least two of the ten genes (Table 1) can be used as a proxy for Moleculeᵢ. Moleculeᵢ can be determined by a person skilled in the art using e.g. RNAseq quantification (relative abundance of each transcript as fragments per kilobase of transcript per million reads FPKM). Moleculeᵢ is then log2 transformed and divided by the SD factor defined in Table 2, and the ViRP-score calculated using the formula with Aᵢ weights as listed in Table 1.

In an alternative embodiment of the method, phosphorylated proteins or unphosphorylated proteins corresponding to at least two of the nine proteins (Table 1) can be used as a proxy for Moleculeᵢ. In the current predictor model according to Table 1, protein number 1, 5 and 6, the level of phosphorylated protein is quantitated. Moleculeᵢ can be determined by a person skilled in the art using e.g. RPPA quantification. Moleculeᵢ is then log2 transformed and divided by the SD factor defined in Table 2, and the ViRP-score calculated using the formula with Aᵢ weights as listed in Table 1.

When based on normalized expression levels, the selected ViRP cutoff value may be between 40-50. For example, in a dataset where relative expression of each gene is log2 transformed, then divided by a factor SD defined in Table 2 and the transformed to a score range 0-100 using the formula:

$$\text{ViRPscore} = \frac{\text{ViRPscore} - min \left(\text{ViRPscore}\right)}{max \left(\text{ViRPscore}\right) - min \left(\text{ViRPscore}\right)} * 100$$

TABLE 2

SD correction factor for each protein/gene in signature

| | Protein | Gene | SD |
|---|---|---|---|
| 1 | ACC-pS79 | ACACA/ ACACB | 0.59 |
| 2 | Bc12 | BCL2 | 0.81 |
| 3 | Chk1 | CHEK1 | 0.51 |
| 4 | Fibronectin | FN1 | 0.63 |
| 5 | Myosin Iia-pS1943 | MYH9 | 0.49 |
| 6 | NDRG1-pT346 | NDRG1 | 1.00 |
| 7 | Notch1 | NOTCH1 | 0.40 |
| 8 | p21 | CDKN1A | 0.56 |
| 9 | Syk | SYK | 0.43 |
| 10 | TP53BP1 | TP53BP1 | 0.55 |

In some embodiments of the above, the selected cutoff value is from about 40 to about 45 or alternatively from about 45 to about 50. Exemplary cutoff values are shown in FIGS. 5A, C and FIG. 6. A skilled person is able to determine, based on the receiver operating characteristic (ROC) curves, an appropriate cutoff value based on an acceptable number of true positives vs false positives.

Information about patients who will have a positive clinical response of receiving a VEGF-A inhibitory drug can be used in treatment strategies for cancer patients. For example, patients likely to have a beneficial clinical effect of a VEGF-A inhibitory drug can be treated while patients less likely to respond to the treatment can be spared. The latter patients may thus avoid adverse side effects of the treatment and can instead be considered for other types of treatment.

Furthermore, the inventors have established a predictor model based on a novel combination of known molecular, clinical and statistical methods, wherein the predictor model is able to identify proteomic variables that is associated with a predictive clinical effect of a VEGF-A inhibitory drug from the proteomic information.

Importantly, the predictor model is only dependent on a relatively small number of genes wherein the mRNA or proteins encoded by the genes can be obtained from a sample isolated by routine methods from the cancer patients.

An additional advantage of the method is that a pre-defined panel of proteins known to be involved in cancer and with adequately detectable variations in expression levels is used as input to train the predictor model, and not just an arbitrary selection derived from all known genes.

Furthermore, the predictor model is based on a two-step process for selecting the final set of biomarkers and corresponding optimized coefficients. This two-step process gives a more precise model compared to a single step method.

Based on the unique official gene and protein identifiers given in Table 3 below, one skilled in the art can readily obtain antibodies, primers and/or probes that can be used in the measuring of the expression level of genes in Table 1.

TABLE 3

Proteins and protein encoding genes with reference numbers to protein and nucleic acid sequences and antibodies for detection of proteins or phosphorylated proteins

| | Protein | Gene | GCID | HGNC | UniProtKB | Antibody |
|---|---|---|---|---|---|---|
| 1 | ACC-pS79 | ACACA/ ACACB | GC17M037084/ GC12P109116 | HGNC:84/ HGNC:85 | Q13085/ 000763 | CST: #3661 |
| 2 | Bc12 | BCL2 | GC18M063123 | HGNC:990 | P10415 | CST: #3498 |
| 3 | Chk1 | CHEK1 | GC11P125625 | HGNC:1925 | 014757 | CST: #2360 |
| 4 | Fibronectin | FN1 | GC02M215360 | HGNC:3778 | P02751 | Abcam: ab45688 |
| 5 | Myosin Iia-pS1943 | MYH9 | GC22M036281 | HGNC:7579 | P35579 | CST: #5026 |
| 6 | NDRG1-pT346 | NDRG1 | GC08M133237 | HGNC:7679 | Q92597 | CST: # 3217 |
| 7 | Notch1 | NOTCH1 | GC09M136558 | HGNC:7881 | P46531 | CST: #3608 |
| 8 | p21 | CDKN1A | GC06P043169 | HGNC:1784 | P38936 | Santa Cruz: sc-397 |
| 9 | Syk | SYK | GC09P091055 | HGNC:11491 | P43405 | Santa Cruz: sc-1240 |
| 10 | TP53BP1 | TP53BP1 | GC15M043403 | HGNC:11999 | Q12888 | CST: #4937 |

One way to use the ViRP-score according to the present invention is in determining if a patient is likely to have a favorable clinical effect of receiving a pharmaceutical composition comprising a VEGF-A-inhibitor is to generate a ViRP cutoff value or threshold value. Such cutoff value can be used to predict if a cancer patient will benefit from receiving a pharmaceutical composition comprising a VEGF-A-inhibitor or not wherein a ViRP-score being lower than a selected cutoff ViRP-value is indicative of the patient being responsive to a composition comprising a VEGF-A inhibitor drug.

The exact cutoff value will vary dependent on which quantification method is used. However, a skilled person would, by performing the method described herein, be able to determine the cutoff value based on retrospective analysis of tumor samples from cancer patients to calculate the ViRP-scores on the selected platform and perform ROC analysis to determine the best cutoff value for balancing the number of true and false positives, or select a clinical appropriate cutoff to encompass a higher number of potential responders.

In the following example, the cutoff value for assigning a cancer patient into the low ViRP group that will benefit from receiving treatment is defined as having a ViRP score below 48.5, see ROC curves in FIGS. 5A and C for examples based on protein expression level and FIG. 7 for examples of increase in patients responding to treatment when using this cutoff. In another example the cutoff value for assigning a cancer patient into the low ViRP group that will benefit from receiving treatment is defined as having a ViRP-score below 46.8 and 47.3, see ROC curves in FIGS. 6A and C for examples based on mRNA expression levels.

### Treatment Regimen

The patients diagnosed with a malignant solid tumor and wherein the ViRP-score predicts that they will benefit from a treatment regimen comprising a VEGF-A-inhibitory drug, may in one embodiment of the disclosure be given the VEGF-A -inhibitory drug in a neoadjuvant treatment setting. A neoadjuvant treatment setting according to the present disclosure is a regimen where the malignant tumor is removed after the VEGF-A -inhibitory drug is administrated to the patient over a time period (e.g. 24 weeks).

In an alternative embodiment of the invention, the VEGF-A -inhibitory drug is given in an adjuvant treatment setting. Adjuvant treatment is a therapy where the patient is given an additional treatment over a period after the malignant tumor has been surgically removed. In a further embodiment of the invention the cancer patients are given chemotherapy in addition to a VEGF-A -inhibitory drug.

The VEGF-A-inhibitory drug may in an alternative embodiment of the disclosure be an anti-VEGF-A antibody. The anti-VEGF-A antibody may in an alternative embodiment of the disclosure be the monoclonal antibody bevacizumab (abbreviation: Bev) sold under the trade name Avastin (Roche), Mvasi (Amgen) or Zirabev (Pfizer).

Chemotherapy (abbreviation: CTx) is a treatment that uses drugs to stop the growth of cancer cells, either by killing the cells or by stopping them from dividing. Chemotherapy may be administered orally, topically or parenterally (e.g. via intravenous infusions or subcutaneous injections), depending on the type and stage of the cancer being treated. It may be given alone or with other treatments, such as surgery, radiation therapy, or biologic therapy. Suitable chemotherapeutic agents are known to a skilled person and includes among other commonly used agents such as anthracyclines, taxanes or drugs derived from platinum salts.

As used herein, a subject “eligible for treatment with neoadjuvant chemotherapy” means a patient who has an invasive breast cancer that by a skilled person is evaluated to benefit from neoadjuvant chemotherapy. Such patients may have tumors that are eligible based on an evaluation of aggressiveness, size or a presentation and distribution of the disease that make neoadjuvant therapy the best choice for an optimal treatment result.

The NeoAVA study NCT00773695 is a study of bevacizumab (Avastin) in combination with neoadjuvant treatment regimens in participants with primary human epidermal growth factor receptor 2 (HER2) negative breast cancer.

The PROMIX study NCT00957125 is a Translational Trial on Molecular Markers and Functional Imaging to Predict Response of Preoperative Treatment of Breast Cancer Early (PROMIX). Patients with localized primary breast cancer including inflammatory breast cancer suitable for primary medical treatment and/or regional lymph node metastases receive six cycles of chemotherapy with epirubicin and docetaxel. Treatment evaluations are performed after the second, fourth and sixth cycle. In case of SD/PR after the second course, bevacizumab is added to the combination for the remaining four courses.

### Relative Importance of Proteins and Modelling of Protein Combinations

Ranking the relative importance of the ten original genes using the R-package “Relaimpo” demonstrate that some genes may contribute more to the predictive ability to significantly separate the pCR from non-pCR responding patients, see FIG. 12. With reference to FIG. 12, for example the Syk and Myosin IIa-pS1943 proteins is of high importance, thus a combination of these two or the top five genes (Syk, NDRG1-pT346, p21 and TP53BP1) might be of particular interest.

By combining the original ten genes discovered by Lasso regression in all possible configurations, 1024 (2^10) unique models were created. Omitting the empty model not containing any proteins and the ten models containing only one protein, 1013 possible configurations remained. By analyzing these combinations of genes by Lasso regression coupling protein expression levels to relative tumor size (tumor size after relative to before treatment) a set of 1013 new protein signature score models were obtained. Assessment of all these models for their ability to significantly separate the pCR from non-pCR responding patients using protein or mRNA expression levels as input demonstrated that numerous models even with only two out of ten proteins, gave significant separation (p<0.05), for example a model based on the proteins Syk and Notch1. Comparison of this minimal two-gene model on similar parameters as the full ten-protein model demonstrated significant performance on the internal NeoAva and external PROMIX datasets (FIGS. 8, 9 and 10).

The method disclosed herein consistently provides separation of pCR and non-pCR responders with p-value <0.05 and “Area Under the Curve” (AUC) values above 0.5 using either protein or mRNA expression, as exemplified in Table 5. Accordingly, it can be used for prediction whether a subject is responsive to a VEGF-A inhibitory drug.

TABLE 5

Area under the curve (AUC) for models relying on two to ten genes selected from All models give ViRP signature scores significantly separating pCR vs non-pCR patients using either protein or mRNA expression as input (p<0.05)

| Number of genes in model | Gene names | AUC NeoAVA Protein | AUC NeoAVA mRNA | AUC PROMIX mRNA |
|---|---|---|---|---|
| 2 | Syk, p21 | 0.81 | 0.67 | 0.68 |
| 2 | Syk, X53BP1 | 0.77 | 0.69 | 0.69 |
| 2 | NDRG1_pT346, p21 | 0.77 | 0.67 | 0.67 |
| 2 | Syk, Notch1 | 0.74 | 0.75 | 0.64 |
| 2 | Syk, ACC_pS79 | 0.75 | 0.66 | 0.71 |
| 2 | Syk, NDRG1_pT346 | 0.76 | 0.73 | 0.70 |
| 2 | NDRG1_pT346, ACC_pS79 | 0.71 | 0.66 | 0.74 |
| 3 | Syk, ACC_pS79, p21 | 0.82 | 0.68 | 0.69 |
| 3 | Syk, X53BP1, p21 | 0.83 | 0.69 | 0.69 |
| 3 | Syk, NDRG1_pT346, p21 | 0.82 | 0.73 | 0.69 |
| 3 | Syk, Notchl, p21 | 0.80 | 0.77 | 0.64 |
| 3 | Syk, NDRG1_pT346, ACC_pS79 | 0.78 | 0.73 | 0.74 |
| 3 | Syk, Notchl, ACC_pS79 | 0.76 | 0.74 | 0.66 |
| 3 | Bc12, NDRG1_pT346, X53BP1 | 0.72 | 0.69 | 0.71 |
| 3 | Fibronectin, Bc12, ACC_pS79 | 0.71 | 0.64 | 0.71 |
| 3 | Chk1, Notchl, NDRG1_pT346 | 0.71 | 0.71 | 0.68 |
| 3 | Chk1, Fibronectin, Bc12 | 0.70 | 0.69 | 0.68 |
| 3 | Bc12, NDRG1_pT346, ACC_pS79 | 0.73 | 0.66 | 0.74 |
| 3 | Chk1, Bc12, NDRG1_pT346 | 0.71 | 0.69 | 0.67 |
| 3 | Bc12, X53BP1, ACC_pS79 | 0.69 | 0.64 | 0.68 |
| 4 | Syk, X53BP1, ACC_pS79, p21 | 0.82 | 0.69 | 0.70 |
| 4 | Syk, NDRG1_pT346, ACC_pS79, p21 | 0.82 | 0.72 | 0.71 |
| 4 | Syk, Notchl, ACC_pS79, p21 | 0.81 | 0.74 | 0.66 |
| 4 | Syk, Bc12, ACC_pS79, p21 | 0.78 | 0.71 | 0.71 |
| 4 | Chk1, Syk, ACC_pS79, p21 | 0.85 | 0.71 | 0.72 |
| 4 | Syk, NDRG1_pT346, X53BP1, p21 | 0.82 | 0.74 | 0.70 |
| 4 | Syk, NDRG1_pT346, X53BP1, ACC_pS79 | 0.80 | 0.75 | 0.74 |
| 4 | Chk1, Myosin_IIa_pS1943, Syk, NDRG1_pT346 | 0.87 | 0.74 | 0.66 |
| 4 | Fibronectin, Syk, Bc12, ACC_pS79 | 0.75 | 0.66 | 0.75 |
| 4 | Chk1, Fibronectin, Bc12, ACC_pS79 | 0.77 | 0.69 | 0.75 |
| 5 | Fibronectin, Syk, X53BP1, ACC_pS79, p21 | 0.78 | 0.68 | 0.71 |
| 5 | Chk1, Syk, X53BP1, ACC_pS79, p21 | 0.86 | 0.73 | 0.72 |
| 5 | Syk, Notch1, NDRG1_pT346, ACC_pS79, p21 | 0.82 | 0.78 | 0.70 |
| 5 | Syk, Bc12, NDRG1_pT346, ACC_pS79, p21 | 0.80 | 0.71 | 0.71 |
| 5 | Chk1, Myosin_IIa_pS1943, Syk, ACC_pS79, p21 | 0.89 | 0.71 | 0.71 |
| 5 | Chk1, Fibronectin, Syk, ACC_pS79, p21 | 0.84 | 0.69 | 0.73 |
| 5 | Syk, Notch1, NDRG1_pT346, X53BP1, p21 | 0.82 | 0.78 | 0.68 |
| 5 | Syk, Bc12, NDRG1_pT346, X53BP1, p21 | 0.81 | 0.72 | 0.7 |
| 6 | Chk1, Syk, Notchl, X53BP1, ACC_pS79, p21 | 0.86 | 0.73 | 0.72 |
| 6 | Chk1, Syk, Bc12, X53BP1, ACC_pS79, p21 | 0.84 | 0.74 | 0.72 |
| 6 | Chk1, Myosin_IIa_pS1943, Bc12, X53BP1, ACC_pS79, p21 | 0.81 | 0.71 | 0.68 |
| 6 | Fibronectin, Myosin_IIa_pS1943, Syk, X53BP1, ACC_pS79, p21 | 0.81 | 0.67 | 0.67 |
| 6 | Chk1, Myosin_IIa_pS1943, Syk, X53BP1, ACC_pS79, p21 | 0.88 | 0.74 | 0.71 |
| 6 | Chk1, Fibronectin, Syk, | 0.83 | 0.71 | 0.74 |
| | X53BP1, ACC_pS79, p21 | | | |
| 6 | Chk1, Fibronectin, Bc12, NDRG1_pT346, X53BP1, ACC_pS79 | 0.82 | 0.7 | 0.76 |
| 6 | Fibronectin, Myosin_IIa_pS1943, Syk, NDRG1_pT346, X53BP1, ACC_pS79 | 0.84 | 0.67 | 0.72 |
| 6 | Chk1, Myosin_IIa_pS1943, Syk, NDRG1_pT346, X53BP1, ACC_pS79 | 0.88 | 0.75 | 0.71 |
| 7 | Myosin_IIa_pS1943, Syk, Notchl, NDRG1_pT346, X53BP1, ACC_pS79, p21 | 0.86 | 0.79 | 0.67 |
| 7 | Chk1, Myosin_IIa_pS1943, Notchl, NDRG1_pT346, X53BP1, ACC_pS79, p21 | 0.83 | 0.71 | 0.70 |
| 7 | Chk1, Fibronectin, Notchl, NDRG1_pT346, X53BP1, ACC_pS79, p21 | 0.82 | 0.70 | 0.76 |
| 7 | Chk1, Myosin_IIa_pS1943, Syk, Notchl, NDRG1_pT346, X53BP1, ACC_pS79 | 0.88 | 0.76 | 0.71 |
| 7 | Chk1, Fibronectin, Syk, Notchl, NDRG1_pT346, X53BP1, ACC_pS79 | 0.83 | 0.72 | 0.76 |
| 7 | Chk1, Fibronectin, Myosin_IIa_pS1943, Notchl, NDRG1_pT346, X53BP1, ACC_pS79 | 0.83 | 0.70 | 0.74 |
| 7 | Fibronectin, Myosin_IIa_pS1943, Syk, Bc12, NDRG1_pT346, X53BP1, ACC_pS79 | 0.83 | 0.68 | 0.71 |
| 7 | Chk1, Myosin_IIa_pS1943, Syk, Bc12, NDRG1_pT346, X53BP1, ACC_pS79 | 0.87 | 0.75 | 0.70 |
| 8 | Chk1, Fibronectin, Syk, Bc12, Notchl, NDRG1_pT346, X53BP1, ACC_pS79 | 0.83 | 0.72 | 0.76 |
| 8 | Chk1, Fibronectin, Myosin_IIa_pS1943, Bc12, Notchl, NDRG1_pT346, X53BP1, ACC_pS79 | 0.84 | 0.70 | 0.74 |
| 8 | Chk1, Fibronectin, Myosin_IIa_pS1943, Syk, Notchl, NDRG1_pT346, X53BP1, ACC_pS79 | 0.86 | 0.73 | 0.74 |
| 8 | Chk1, Fibronectin, Myosin_IIa_pS1943, Syk, Bc12, NDRG1_pT346, X53BP1, ACC_pS79 | 0.86 | 0.72 | 0.74 |
| 8 | Chk1, Fibronectin, Myosin_IIa_pS1943,Syk, Bc12, Notch1, X53BP1, ACC_pS79 | 0.84 | 0.73 | 0.72 |
| 8 | Chk1, Fibronectin, Myosin_IIa_pS1943, Syk, Bc12, Notch1, NDRG1_pT346, ACC_pS79 | 0.85 | 0.72 | 0.74 |
| 8 | Chk1, Fibronectin, Myosin_IIa_pS1943, Syk, Bc12, Notch1, NDRG1_pT346, X53BP1 | 0.84 | 0.74 | 0.69 |
| 9 | Fibronectin, Myosin_IIa_pS1943, Syk, Bc12, Notch1, NDRG1_pT346, X53BP1, ACC_pS79, p21 | 0.84 | 0.69 | 0.71 |
| 9 | Chk1, Myosin_IIa_pS1943, Syk, Bc12, Notch1, NDRG1_pT346, X53BP1, ACC_pS79, p21 | 0.87 | 0.75 | 0.70 |
| 9 | Chk1, Fibronectin, Syk, Bc12, Notch1, NDRG1_pT346, X53BP1, ACC_pS79, p21 | 0.84 | 0.72 | 0.75 |
| 9 | Chk1, Fibronectin, Myosin_IIa_pS1943, Bc12, Notch1, NDRG1_pT346, X53BP1, ACC_pS79, p21 | 0.83 | 0.70 | 0.74 |
| 9 | Chk1, Fibronectin, Myosin_IIa_pS1943, Syk, | 0.86 | 0.73 | 0.74 |
| | Notch1, NDRG1_pT346, X53BP1, ACC_pS79, p21 | | | |
| 9 | Chk1, Fibronectin, Myosin_IIa_pS1943, Syk, Bc12, NDRG1_pT346, X53BP1, ACC_pS79, p21 | 0.86 | 0.72 | 0.74 |
| 9 | Chk1, Fibronectin, Myosin_IIa_pS1943, Syk, Bc12, Notch1, X53BP1, ACC_pS79, p21 | 0.84 | 0.73 | 0.71 |
| 9 | Chk1, Fibronectin, Myosin_IIa_pS1943, Syk, Bc12, Notch1, NDRG1_pT346, ACC_pS79, p21 | 0.85 | 0.72 | 0.73 |
| 9 | Chk1, Fibronectin, Myosin_IIa_pS1943, Syk, Bc12, Notch1, NDRG1_pT346, X53BP1, p21 | 0.83 | 0.74 | 0.69 |
| 9 | Chk1, Fibronectin, Myosin_IIa_pS1943, Syk, Bc12, Notch1, NDRG1_pT346, X53BP1, ACC_pS79 | 0.86 | 0.73 | 0.74 |
| 10 | Chk1, Fibronectin, Myosin_IIa_pS1943, Syk, Bc12, Notch1, NDRG1_pT346, X53BP1, ACC_pS79, p21 | 0.85 | 0.73 | 0.74 |

### EXAMPLES

The following examples are for illustration purpose only.

### Example 1: Identification of Ten Protein Prognostic Signature

The following illustrates how the ten protein prognostic signature was identified and how it can be used in determining which patients will gain a positive clinical outcome of a treatment comprising a VEGF-A-inhibitory drug in a clinical laboratory testing.

Tumors from HER2-negative breast cancer patients were used in the examples described below. An initial set of 210 cancer related proteins were analyzed.

### Patient Cohort

138 patients with HER2-negative previously untreated breast carcinomas with size ≥2.5 cm was included, and 67 and 71 patients randomized to treatment with CTx (chemotherapy) or Bev+CTx (chemotherapy in combination with bevacizumab), respectively. A response evaluation to antiangiogenic neoadjuvant treatment and protein expression profiles were established in the NeoAva phase II clinical trial (NeoAva-NCT00773695).

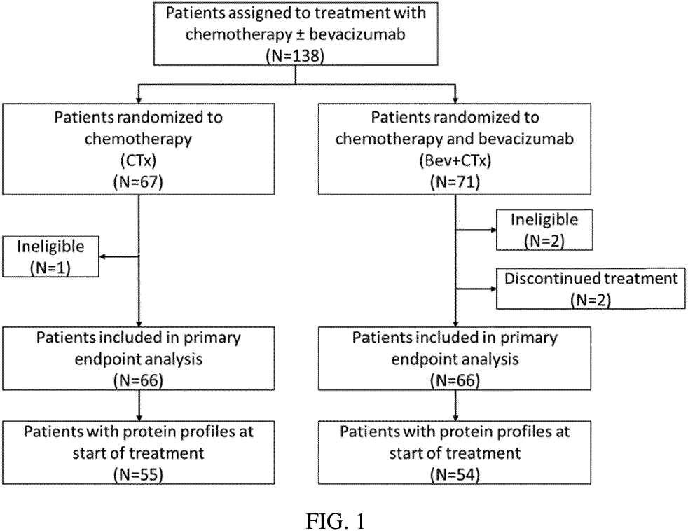

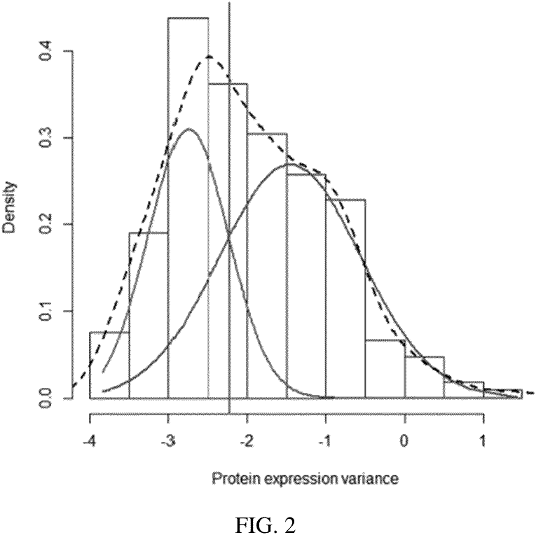

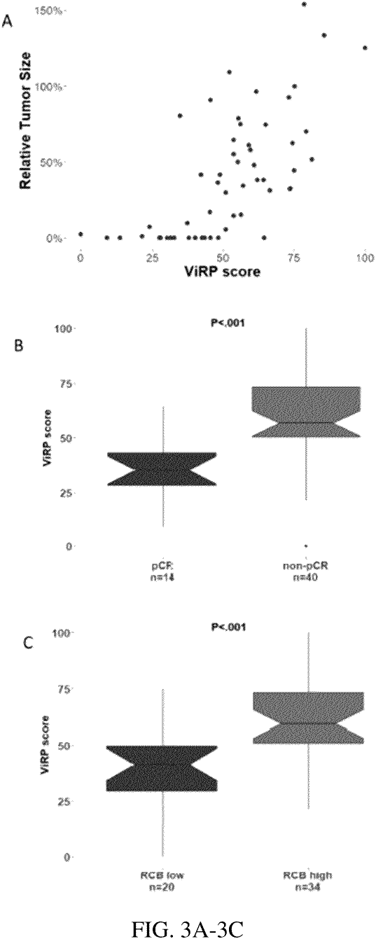

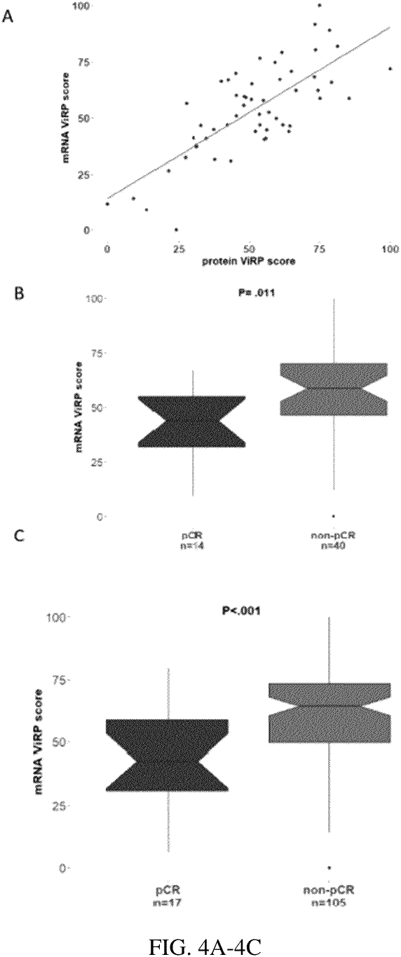

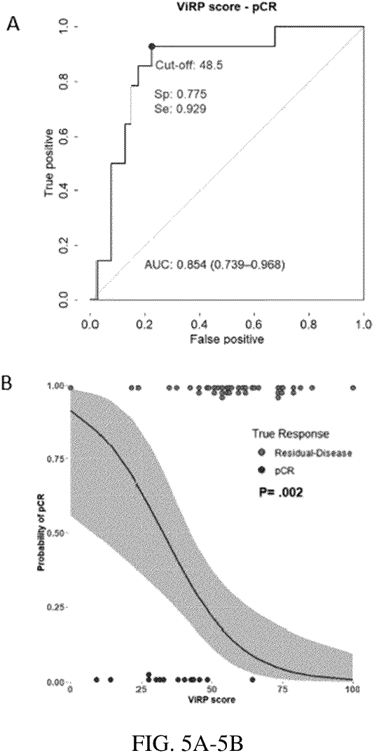

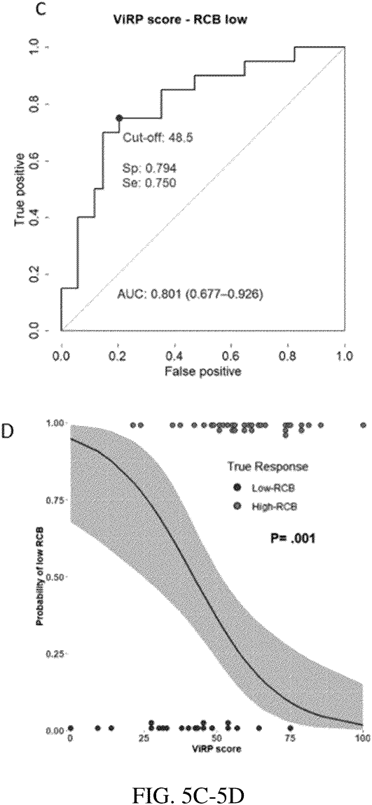

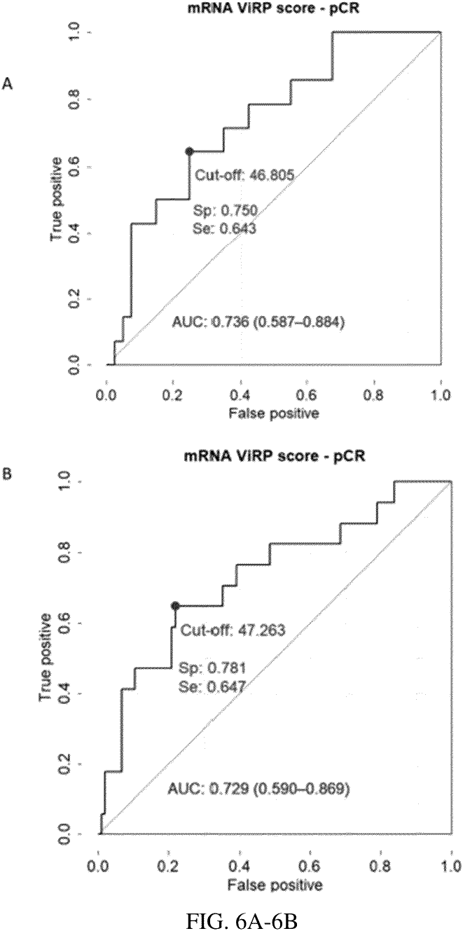

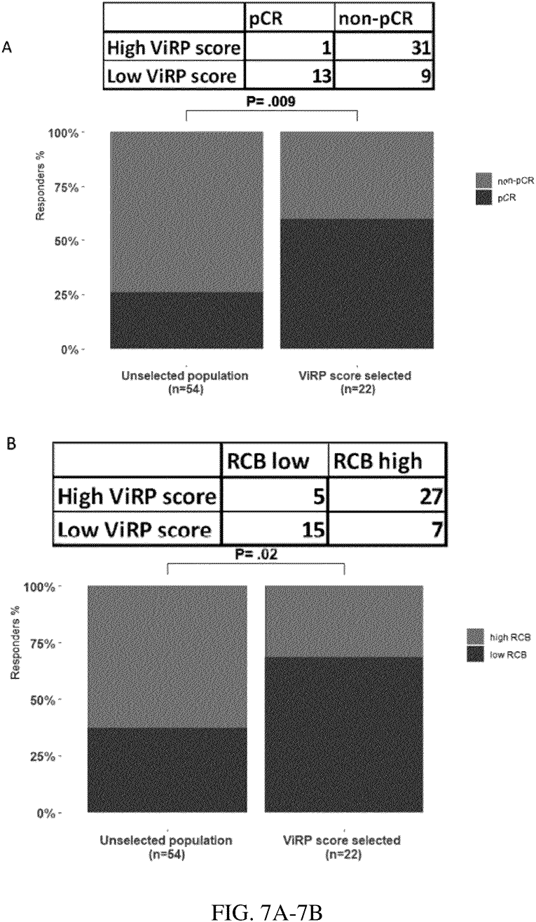

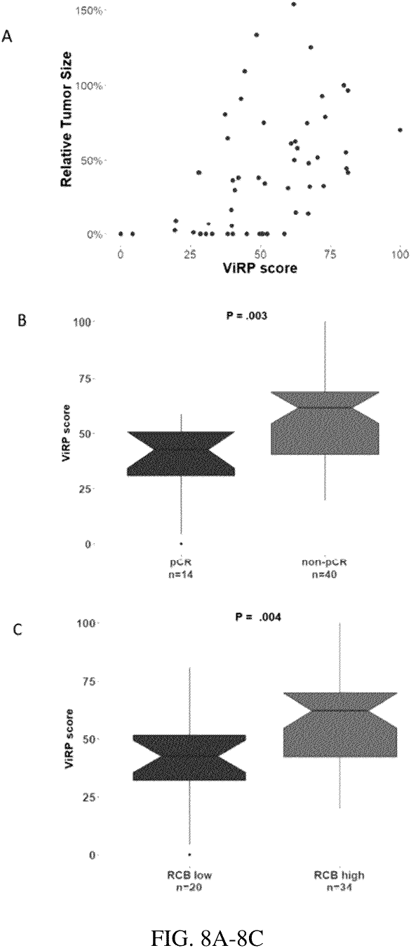

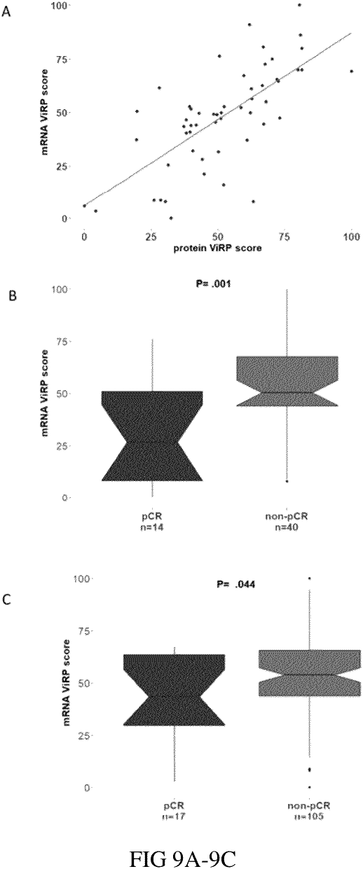

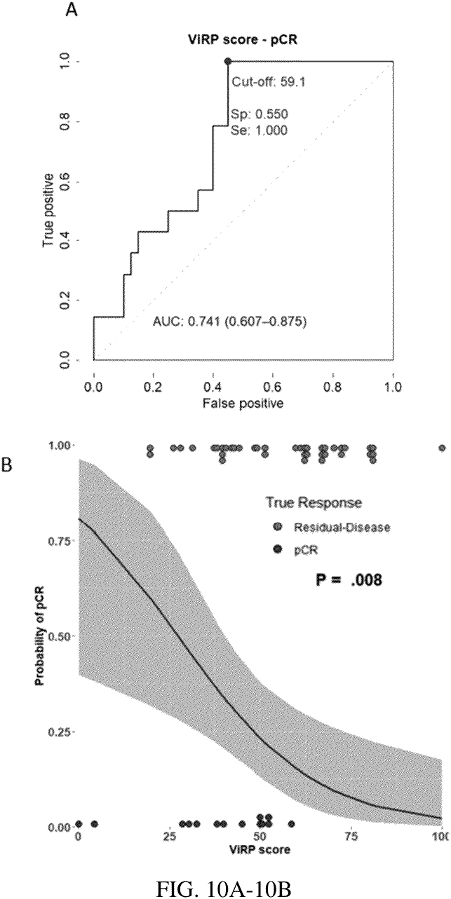

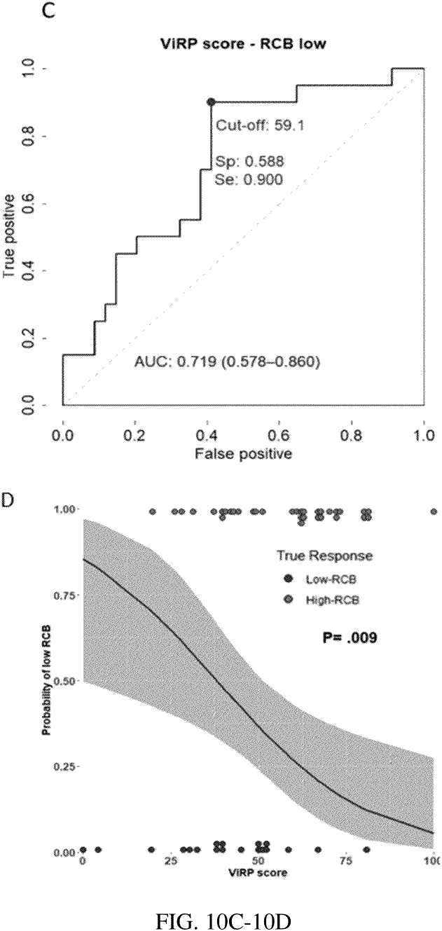

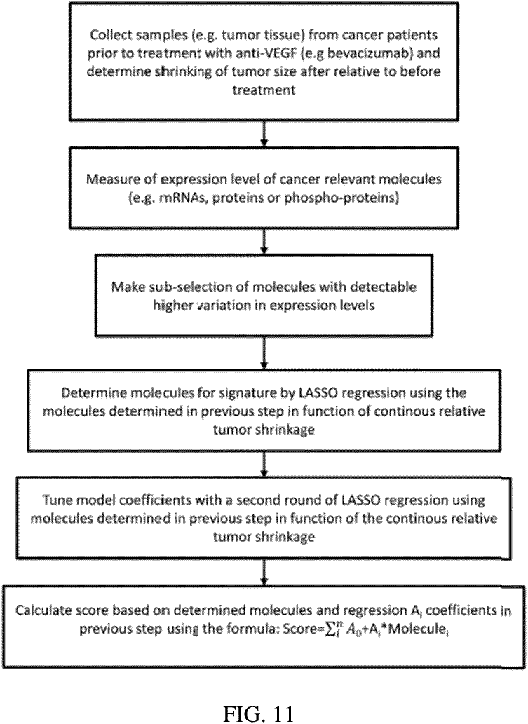

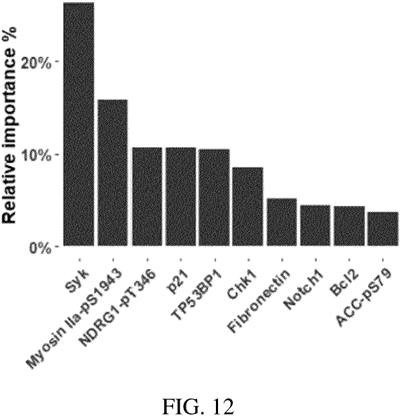

In each treatment arm, 66 patients were included in the primary endpoint analysis (pCR) and a total of 109 samples were analyzed on RPPA with protein profiles prior to treatment obtained for patients in the CTx (n=55) and Bev+CTx (n=54) treatment arms (FIG. 1). Clinicopathological characteristics of patients, including adverse events have previously been described in relation to mRNA expression (Silwal-Pandit L, et al: The Longitudinal Transcriptional Response to Neoadjuvant Chemotherapy with and without Bevacizumab in Breast Cancer. Clinical Cancer Research 2017, vol.23, p.4662-4670). In brief the primary endpoint, pCR, was defined as pathologic stage ypT0 and ypN0 after ended therapy. pCR rates were overall higher in the Bev+CTx (23 %) compared the CTx (12 %) treatment arm, although not significantly (P=0.17). Within the ER-positive subset a significantly (P=0.02) higher pCR rate was observed in the Bev+CTx (20 %) compared to the CTx (5 %) treatment arm. In addition to pCR, response to neoadjuvant treatment was evaluated by relative tumor size after 24 weeks, calculated as the percentage of tumor size at time of surgery (longest diameter on histopathological specimen) relative to tumor size at week 0 (MRI if available or Ultrasound/Mammography). RCB was calculated using the Residual Cancer Burden Calculator (MD Anderson Cancer Center)(Symmans WF, et al: Measurement of Residual Breast Cancer Burden to Predict Survival After Neoadjuvant Chemotherapy. Journal of Clinical Oncology 2007, vol.25, p.4414-4422), and dichotomized to “low” and “high” for RCB0/I and RCBII/III, respectively. The study was approved by the Institutional Protocol Review Board, the regional ethics committee, the Norwegian Medicines Agency, and performed in accordance with the Declaration of Helsinki. We further used an external cohort with similar treatment characteristics (PROMIX trial NCT00957125) and with mRNA expression data available for validation of the ViRP signature (Kimbung S, et al: Assessment of early response biomarkers in relation to long-term survival in patients with HER2-negative breast cancer receiving neoadjuvant chemotherapy plus bevacizumab: Results from the Phase II PROMIX trial. Int J Cancer 2018, vol.142, p.618-628).

### Sample Processing and Protein Expression Profiling by Reverse Phase Protein Arrays (RPPA)

Profiling of 210 cancer relevant proteins of which 54 were in a phosphorylated state were performed by RPPA (Lu Y et al: Using reverse-phase protein arrays as pharmacodynamic assays for functional proteomics, biomarker discovery, and drug development in cancer. Seminars in Oncology, 2016, vol. 43, p. 476-483) at the core facility at MD Anderson Cancer Center (Houston, TX). Tumor protein lysates were serially diluted two-fold for 5 dilutions (from undiluted to 1:16 dilution) and arrayed on nitrocellulose-coated slides (in an 11×11 format). Samples were probed with antibodies by tyramide-based signal amplification approach, visualized by DAB colorimetric reaction. Slides were scanned on a flatbed scanner to produce 16-bit tiff image and the spot density was quantified by Array-Pro Analyzer. Relative protein levels for each sample were determined by interpolation of each dilution curves from the “standard curve” (SuperCurve) of the slide (antibody). All the data points were normalized for protein loading by using a protein loading correction factor (CF), and CF less than 0.25 or greater than 2.5, we removed as outliers. All the values were Log2 transformed and median centered across each antibody.

### Statistical Analysis and Signature Development

The analyses in this report were conducted using the statistical programming language R (v 3.5.2) (R Foundation for Statistical Computing, Vienna, Austria) with RStudio (v 1.1.463). To assess significance of differences in scores in subgroups, a two-sample t-test was applied. To assess significance of correlations between continuous variables, a Spearman or Pearson correlation test was applied, as indicated. All statistical tests were two-sided and a P-value below .05 was considered significant. Fisher’s exact test was used to compare response (pCR and RCB) between groups.

To develop a molecular signature predicting response to treatment with Bev+CTx, protein expression from tumor needle biopsies taken prior to treatment were used from patients with available tumor size before and after neo-adjuvant treatment (n=54). Low variance proteins, being likely to have low predictive value due to low signal to noise ratio, were filtered out by fitting a mixed model distribution to the protein variances using the R-package “mixtools” (Benaglia T, et al: mixtools: An R Package for Analyzing Mixture Models. Journal of Statistical Software; 2009, Vol 1, Issue 6), and discarding proteins in the lower cluster i.e. to the left of the vertical line (FIG. 2). Adaptive Lasso-regression (Least Absolute Shrinkage and Selection Operator) (Tibshirani R: Regression Shrinkage and Selection via the Lasso. Journal of the Royal Statistical Society. Series B (Methodological) 1996, vol.58, p.267-288) was performed using the R-package “glmnet” (Friedman JH, Hastie T, Tibshirani R: Regularization Paths for Generalized Linear Models via Coordinate Descent.

Journal of Statistical Software; 2010, Vol 1, Issue 1) in which the penalty parameter lambda was determined by cv.glmnet using leave-one-out cross-validation with mean absolute error loss, and the lambda.min values selected. ROC curves were analyzed using the R-package “pROC” (Robin X, Turck N, Hainard A, et al: pROC: an open-source package for R and S+ to analyze and compare ROC curves. BMC Bioinformatics, 2011, vol. 12, p.77).

ViRP-scores based on mRNA data in the NeoAva and PROMIX study were calculated using the intercept and beta-coefficients determined from the protein data from the NeoAva trial. The corresponding surrogate mRNA ViRP-scores were determined using probe averaged and quantile normalized mRNA expressions from the genes corresponding to the proteins in the original protein signature, including the phospho-proteins.

### Development of a Predictive Protein Signature Score

A certain level of variance in expression should be present in order for clinical markers to be applicable for use in signatures. We thus reduced the original panel of 210 proteins by plotting the variance in expression and demonstrated a mixed distribution (FIG. 2) where only members belonging mainly to the proteins with higher variance (n=114) were considered for use in the adaptive Lasso regression model. By applying Lasso regression coupling protein expression to relative tumor size (post-neoadjuvant treatment), a set of ten proteins with non-zero beta-coefficients were discovered (Table 1). A second iterative round of Lasso on this subset of ten proteins gave the final intercept and beta-coefficients, of which one was shrunk to zero giving a final signature of nine proteins with non-zero Aᵢ coefficients: Chk1, Fibronectin, Myosin_IIa_pS1943,Syk, Bc12, Notchl, NDRG1_pT346, X53BP1 and ACC_pS79

The ViRP-score for each patient was calculated as the sum of the intercept and beta-coefficient weighted expression of the ten proteins. The ViRP-score demonstrated significant and high correlation (Pearson R2=0.67, P=3e-08) with relative tumor size (FIG. 3A). Using pCR as response criteria the ViRP-scores were significantly (P=1.e-04) lower in the pCR compared to non-pCR patients (FIG. 3B). We further evaluated the ViRP-score using RCB class as response criteria, as this has been suggested to provide additional and independent prognostic information to yp stage (Loibl S, Denkert C: How Much Information Do We Really Need After Neoadjuvant Therapy for Breast Cancer? Journal of Clinical Oncology, 2017, vol. 35, p.1029-1030) and has been associated to long term prognostic risk after CTx neoadjuvant (Symmans WF et al: Long-Term Prognostic Risk After Neoadjuvant Chemotherapy Associated With Residual Cancer Burden and Breast Cancer Subtype. Journal of Clinical Oncology, 2017, vol. 35, p.1049-1060). Correlation between the ViRP-scores and the continuous RCB scores was significant (Pearson R²=0.39 P=3e-03) and patients having low RCB (class 0 or I) had significantly (P=1e-04) lower ViRP-scores than patients with high RCB (class II or III) (FIG. 3C).

We next sought to establish and evaluate mRNA as a surrogate. By taking the corresponding genes from the ViRP (including phospho-proteins) and using the pre-defined Lasso intercept and beta-coefficients, the mRNA score for the NeoAva patients (mRNA ViRP-scores) was calculated. Correlation between the mRNA ViRP-scores and the original protein ViRP-scores in all the 54 evaluated patients was highly significant (Pearson R²=0.75 P=5e-15) (FIG. 4A), and the mRNA ViRP-scores related to pCR also demonstrated a significantly (P=1.1e-02) lower score in the pCR compared to non-pCR patients (FIG. 4B).

Having settled the use of mRNA as a reasonable surrogate we sought to validate the protein signature in an external mRNA dataset obtained from patients (n=122) in the comparable clinical trial PROMIX. By calculating the mRNA ViRP-score in the PROMIX patients and relating it to pCR response, we confirmed a significantly (P=5e-04) lower mRNA VIRP-scores in the pCR versus non-pCR (FIG. 4C).

### Predictive Performance of VIRP-Scores

The predictive performance of the ViRP-score was evaluated using receiver operating characteristic (ROC) curves. The predictive accuracy (AUC) of the ViRP-score for pCR and low RCB was 0.85 (CI 0.74-0.97) and 0.80 (CI 0.68-0.93), respectively (FIGS. 5A and 5C). Similar results were obtained assessing the mRNA VIRP-scores in NeoAva and PROMIX, demonstrating AUCs of 0.74 (CI 0.59-0.89) and 0.73 (CI 0.59-0.87), respectively (FIG. 6). Additionally, using binomial modelling in a reverse approach, the ViRP-score was evaluated for its ability to predict the probability of pCR or low RCB (FIGS. 5B and 5D), which demonstrated significant results (P=1.9e-03 and P=1.5e-03, respectively).

To assess the potential clinical benefit of using the ViRP-score to select patients for treatment with Bev+CTx, the score value determined by ROC analysis for optimal balance between true and false pCR was used as cutoff (48.5). Fisher’s tables showed significant enrichment of responders for both pCR (P=5e-06) and low RCB (P=1e-04). Furthermore, the fraction of patients responding by pCR or low RCB was compared in the unselected versus the ViRP-score selected patient population. This demonstrated a significant increase approximately doubling in the percentage of both pCR responders (P=9e-03) and low RCB (P=2e-02) in the ViRP-score selected patient population in the NeoAva trial (FIG. 7).

The present inventors have defined a protein signature score named VEGF inhibitory Response Predictor (ViRP) for use in large Her-2 negative primary breast cancers that identifies good responders to neo-adjuvant treatment with chemotherapy and bevacizumab. Use of the ViRP-score for patient selection to Bev+CTx therapy, significantly increase and approximately double, the pCR and RCB-low rate. Furthermore, the robustness of the ViRP-score was confirmed through validation in an independent clinical cohort (the PROMIX study).

In the exemplary embodiments, various features and details are shown in combination. The fact that several features are described with respect to a particular example should not be construed as implying that those features by necessity have to be included together in all embodiments of the disclosure. Conversely, features that are described with reference to different embodiments should not be construed as mutually exclusive. As those with skill in the art will readily understand, embodiments that incorporate any subset of features described herein and that are not expressly interdependent have been contemplated by the inventor and are part of the intended disclosure. However, explicit description of all such embodiments would not contribute to the understanding of the principles of the invention, and consequently some permutations of features have been omitted for the sake of simplicity or brevity.

1. An in vitro method for predicting whether a subject diagnosed with a solid malignant tumor is responsive to a VEGF-A (vascular endothelial growth factor A) inhibitory drug, wherein the method comprises the steps:
a. providing a sample comprising cancer cells obtained from the subject;
b. analyzing the sample from step a. by measuring an expression level of at least two genes selected from SYK, NOTCH1, ACACA/ACACB, TP53BP1, CDKN1A, CHEK1, BCL2, MYH9, FN1 and NDRG1;
c. calculating a ViRP (VEGF inhibition Response Predictor) score based on the expression level of the selected genes in step b.; and
d. comparing said ViRP-score calculated in step c. to a selected cutoff ViRP-value,
wherein a ViRP-score lower than the selected cutoff ViRP-value is predicting that the subject is responsive to a VEGF-A inhibitor drug.

2. The method of claim 1, wherein the expression level is measured for at least three, four, five, six, seven or eight genes selected from the genes in step b.

3. The method of claim 1, wherein the expression level is measured for 9 genes selected from the genes in step b.

4. The method of claim 1, wherein the expression level is measured for all ten genes selected from the genes in step b.

5. The method of claim 1, wherein the expression levels are measured by mRNA or protein.

6. The method of claim 1, wherein the expression levels of said genes are normalized.

7. The method of claim 1, wherein the selected cutoff value is from about 40 to about 50.

8. The method of claim 1, wherein the ViRP-score is calculated as the weighted sum of the expression levels adjusted with the respective coefficients in Table 1.

9. The method of claim 1, wherein ViRP-score is calculated as the weighted sum of the normalized gene expression levels and adding a constant to generate a ViRP-score, wherein
$$V i R P = {A}_{0} + {\sum }_{i = 1}^{n} {A}_{i} ∗ M o l e c u l {e}_{i} ,$$
where n= is the number of genes, A₀ = 0.32, Moleculeᵢ is the normalized expression level of each gene with corresponding Aᵢ coefficients as found in
TABLE 1
List of ViRP signature proteins and genes encoding said proteins with weights

| | Protein | Gene | Aᵢ coefficient |
|---|---|---|---|
| 1 | ACC_pS79 | ACACA/ACACB | 0.12 |
| 2 | Bc12 | BCL2 | 0.03 |
| 3 | Chk1 | CHEK1 | 0.19 |
| 4 | Fibronectin | FN1 | 0.15 |
| 5 | Myosin_IIa_pS1943 | MYH9 | 0.11 |
| 6 | NDRG1_pT346 | NDRG1 | 0.07 |
| 7 | Notch1 | NOTCH1 | 0.03 |
| 8 | P21 | CDKN1A | 0.00 |
| 9 | Syk | SYK | 0.12 |
| 10 | TP53BP1 | TP53BP1 | 0.08 |

.

10. The method of claim 1, wherein the VEGF-A inhibitory drug is an anti-VEGF-A antibody.

11. The method of claim 10, wherein the anti-VEGF-A antibody is bevacizumab.

12. The method of claim 1, wherein the malignant tumor is a primary malignant tumor or a metastatic malignant tumor selected from the group consisting of breast cancer, colorectal cancer, lung cancer, ovarian cancer, glioblastoma or kidney cancer.

13. The method of claim 1, wherein the malignant tumor is a primary breast cancer or a metastatic breast cancer.

14. The method of claim 1, wherein the sample is a lysate of blood cells comprising cancer cells or a lysate of cells obtained from a malignant tumor biopsy.

15. The method of claim 1, wherein said subject has been diagnosed with breast cancer and is eligible for treatment with neoadjuvant chemotherapy.

16. A method for treatment of a solid malignant tumor in a subject comprising the steps:
a. providing a sample comprising cancer cells obtained from the subject;
b. analyzing the sample from step a. by measuring an expression level of at least two genes selected from SYK, NOTCH1, ACACA/ACACB, TP53BP1, CDKN1A, CHEK1, BCL2, MYH9, FN1 and NDRG1;
c. calculating a ViRP (VEGF inhibition Response Predictor) score based on the expression level of the selected genes in step b; and
d. administering a therapeutically efficient amount of a VEGF-A inhibitory drug to said subject if the calculated ViRP-score is lower than a selected cutoff ViRP-value.

17. The method of claim 16, wherein the expression level is measured for at least three, four, five, six, seven or eight genes selected from the genes in step b.

18. The method of claim 16, wherein the expression level is measured for 9 genes selected from the genes in step b.

19. The method of claim 16, wherein the expression level is measured for all ten genes selected from the genes in step b.

20. The method of claim 16, wherein the expression levels are measured by mRNA or protein.

21. The method of claim 16, wherein the expression levels of said genes are normalized.

22. The method of claim 16, wherein the selected cutoff value is from about 40 to about 50.

23. The method of claim 16, wherein the ViRP-score is calculated as the weighted sum of the expression levels adjusted with the respective coefficients in Table 1.

24. The method of claim 16, wherein ViRP-score is calculated as the weighted sum of the normalized gene expression levels and adding a constant to generate a ViRP-score, wherein
$$V i R P = {A}_{0} + {\sum }_{i = 1}^{n} {A}_{i} ∗ M o l e c u l {e}_{i} ,$$
where n = is the number of genes, A₀ = 0.32, Moleculeᵢ is the normalized expression level of each gene with corresponding Aᵢ coefficients as found in Table 1
TABLE 2
List of ViRP signature proteins and genes encoding said proteins with weights

| | Protein | Gene | Aᵢ coefficient |
|---|---|---|---|
| 1 | ACC_pS79 | ACACA/ACACB | 0.12 |
| 2 | Bc12 | BCL2 | 0.03 |
| 3 | Chk1 | CHEK1 | 0.19 |
| 4 | Fibronectin | FN1 | 0.15 |
| 5 | Myosin_IIa_pS1943 | MYH9 | 0.11 |
| 6 | NDRG1_pT346 | NDRG1 | 0.07 |
| 7 | Notch1 | NOTCH1 | 0.03 |
| 8 | P21 | CDKN1A | 0.00 |
| 9 | Syk | SYK | 0.12 |
| 10 | TP53BP1 | TP53BP1 | 0.08 |

.

25. The method of claim 16, wherein the VEGF-A inhibitory drug is an anti-VEGF-A antibody.

26. The method of claim 25, wherein the anti-VEGF-A antibody is bevacizumab.

27. The method of claim 16, wherein the malignant tumor is a primary malignant tumor or a metastatic malignant tumor selected from the group consisting of breast cancer, colorectal cancer, lung cancer, ovarian cancer, glioblastoma or kidney cancer.

28. The method of claim 16, wherein the malignant tumor is a primary breast cancer or a metastatic breast cancer.

29. The method of claim 16, wherein the sample is a lysate of blood cells comprising cancer cells or a lysate of cells obtained from a malignant tumor biopsy.

30. The method of claim 16, wherein said subject has been diagnosed with breast cancer and is eligible for treatment with neoadjuvant chemotherapy.

31. Kit for use in the method of claim 1, wherein the kit comprises reagents for the measuring of the protein expression levels and/or mRNA expression levels of at least two, three, four, five, six, seven, eight, nine or ten genes from the set of genes comprising SYK, NOTCH1, ACACA/ACACB, TP53BP1, CDKN1A, CHEK1, BCL2, MYH9, FN1 and NDRG1.
